(19) United States
(12) Patent Application Publication
(10) Pub. No.: US 2008/0269903 A1
(43) Pub. Date: Oct. 30, 2008

(54) Intervertebral disc nucleus replacement implants and methods

(75) Inventors: Tom J. Francis, Cordova, TN (US); Jason Eckhardt, Memphis, TN (US)

Correspondence Address: Medtronic Spinal and Biologics;Attn: Noreen Johnson - IP Legal Department, 2600 Sofamor Danek Drive, Memphis, TN, 38132, US

(73) Assignee: Warsaw Orthopedic, Inc.

(21) Appl. No.: 11/789,602

(22) Filed: Apr. 25, 2007

(51) Int. Cl. A61F 2/44

(52) U.S. Cl. 623/17.16; 623/17.12

(57) ABSTRACT

An intervertebral disc nucleus replacement implant for positioning between adjacent vertebrae of a spinal segment comprises opposing superior and inferior end portions substantially aligned along a longitudinal axis and a compressible, elastic body surrounding part of the end portions. Each of the end portions includes a convex outer surface for contacting respective endplates of the adjacent vertebrae. Additionally, the elastic body includes an outer surface, with the implant having an outer periphery comprising the outer surfaces of the end portions and the outer surface of the body. In certain embodiments, the elastic modulus of the body is lower than the elastic modulus of each of the end portions and the body extends outward of the end portions transverse to the longitudinal axis, such that the body is configured to limit the amount of subsidence of the implant relative to the adjacent vertebrae.

The present disclosure broadly concerns nucleus pulposus implants and methods for their implantation. The present disclosure generally relates to elastic and compressive intervertebral disc nucleus replacement implants and methods for their implantation. More specifically, but not exclusively, the present disclosure contemplates elastic and/or compressive nucleus replacement implants configured for minimal access implantation and easy insertion in the intervertebral disc space, and configured to limit the amount of subsidence of the implants.

The intervertebral disc functions to stabilize the spine and to distribute forces between vertebral bodies. A normal disc includes a gelatinous nucleus pulposus surrounded and confined by an annulus fibrosis. Intervertebral discs may be displaced or damaged due to trauma or disease. Disruption of the annulus fibrosis may allow the nucleus pulposus to protrude into the vertebral canal, a condition commonly referred to as a herniated or ruptured disc. The extruded nucleus pulposus may press on a spinal nerve, which may result in nerve damage, pain, numbness, muscle weakness and paralysis. Intervertebral discs may also deteriorate due to the normal aging process. As a disc dehydrates and hardens, the disc space height will be reduced, leading to instability of the spine, decreased mobility and pain.

One way to relieve the symptoms of these conditions is by surgical removal of a portion or all of the intervertebral disc. The removal of the damaged or unhealthy disc may allow the disc space to collapse, which would lead to instability of the spine, abnormal joint mechanics, nerve damage, as well as severe pain. Therefore, after removal of the disc, adjacent vertebrae are typically fused to preserve the disc space.

Several devices exist to fill an intervertebral space following removal of all or part of the intervertebral disc in order to prevent disc space collapse and to promote fusion of adjacent vertebrae surrounding the disc space. Even though a certain degree of success with these devices has been achieved, full motion is typically never regained after such vertebral fusions. Attempts to overcome these problems have led to the development of partial and full intervertebral disc replacements. Many of these devices are complicated and bulky. Thus, such devices require invasive surgical procedures and typically never fully return the full range of motion desired.

A need therefore exists for elastic, compressive nucleus replacement implants.

### BRIEF DESCRIPTION OF THE DRAWINGS

FIG. 1 is a side view of a cross-section of an intervertebral disc including a nucleus pulposus surrounded by an annulus fibrosis.

FIG. 2 is a top view of a nucleus replacement implant.

FIG. 3 is a side view of a nucleus replacement implant according to the embodiment illustrated in FIG. 2.

FIG. 4 is a side view of a nucleus replacement implant.

FIG. 5 is a top view of a nucleus replacement implant according to the embodiment illustrated in FIG. 4.

FIG. 6 is another top view of a nucleus replacement implant according to the embodiment illustrated in FIGS. 4 and 5.

FIG. 7 is a side view of a cross-section of a nucleus replacement implant implanted in the intervertebral disc space.

FIG. 8 is another side view of a cross-section of a nucleus replacement implant according to the embodiment illustrated in FIG. 7.

FIG. 9 is yet another side view of a nucleus replacement implant according to the embodiment illustrated in FIGS. 7 and 8.

FIG. 10 is a side view of a cross-section of a nucleus replacement implant.

FIG. 11 is a top view of a nucleus replacement implant according to the embodiment illustrated in FIG. 10.

FIG. 12 is another top view of a nucleus replacement implant according to the embodiment illustrated in FIGS. 10 and 11.

FIG. 13 is a side view of a cross-section of a nucleus replacement implant.

FIG. 14 is a side view of a cross-section of a nucleus replacement implant.

FIG. 15 is a side view of a cross-section of a nucleus replacement implant.

FIG. 16 is a side view of a cross-section of a nucleus replacement implant.

FIG. 17 is a side view of a cross-section of a nucleus replacement implant.

FIG. 18 is a side view of a cross-section of a nucleus replacement implant.

FIG. 19 is a side view of a nucleus replacement implant.

FIG. 20 is a side view of a cross-section of a nucleus replacement implant.

FIG. 21 is a side view of a cross-section of a nucleus replacement implant.

FIG. 22 is a side view of a cross-section of a nucleus replacement implant.

FIG. 23 is a top view of a nucleus replacement implant according to the embodiment illustrated in FIG. 22.

FIG. 24 is a side view of a cross-section of a nucleus replacement implant.

FIG. 25 is a top view of a nucleus replacement implant according to the embodiment illustrated in FIG. 24.

### DESCRIPTION OF THE ILLUSTRATED EMBODIMENTS

For the purposes of promoting an understanding of the principles of the disclosure, reference will now be made to the embodiments illustrated in the drawings and specific language will be used to describe the same. It will nevertheless be understood that no limitation of the scope of the claims is thereby intended, such alterations and further modifications in the illustrated devices, and such further applications of the principles of the disclosure as illustrated therein, being contemplated as would normally occur to one skilled in the art to which the disclosure relates.

The present disclosure provides prosthetic intervertebral disc nucleus pulposus implants that may fully or partially replace the natural or native nucleus pulposus in mammals, including humans and other animals. In one aspect of the disclosure, implants are provided that are configured for minimal access implantation, easy insertion in the intervertebral disc space, configured to limit the amount of subsidence of the implants, and expected to have some mobility for normal biomechanics. In certain embodiments, the implants of the present disclosure are each wide enough to support adjacent vertebrae and each include a height sufficient to separate the adjacent vertebrae. Additionally, in certain embodiments, the implants are strong yet flexible, and prevent excessive deformation under increasing lateral and/or axial compressive loading.

For example, a nucleus pulposus implant may include a load bearing elastic body partially surrounding superior and inferior end portions or members of a higher elastic modulus material than the elastic body. It should be appreciated that for the purposes of the present disclosure, as the elastic modulus of a material decreases the elasticity of the material increases and vice versa. Additionally, the surface of the elastic body may include cuts, slots, slits and/or pockets to assist in compression of the implant. In other aspects of the disclosure, nucleus pulposus implants having shape memory are configured to allow extensive short-term manual or other deformation without permanent deformation, cracks, tears, breakage or other damage. In such embodiments, the implants can not only pass through a relatively small incision in the annulus fibrosis, but can also substantially fill and conform to the intervertebral disc space. In one form of the disclosure, an implant includes a load bearing elastic body with shape memory having an inner fold to allow for coiling and recoiling, or wrapping and unwrapping of the implant. Methods of making and implanting the implants described herein are also provided.

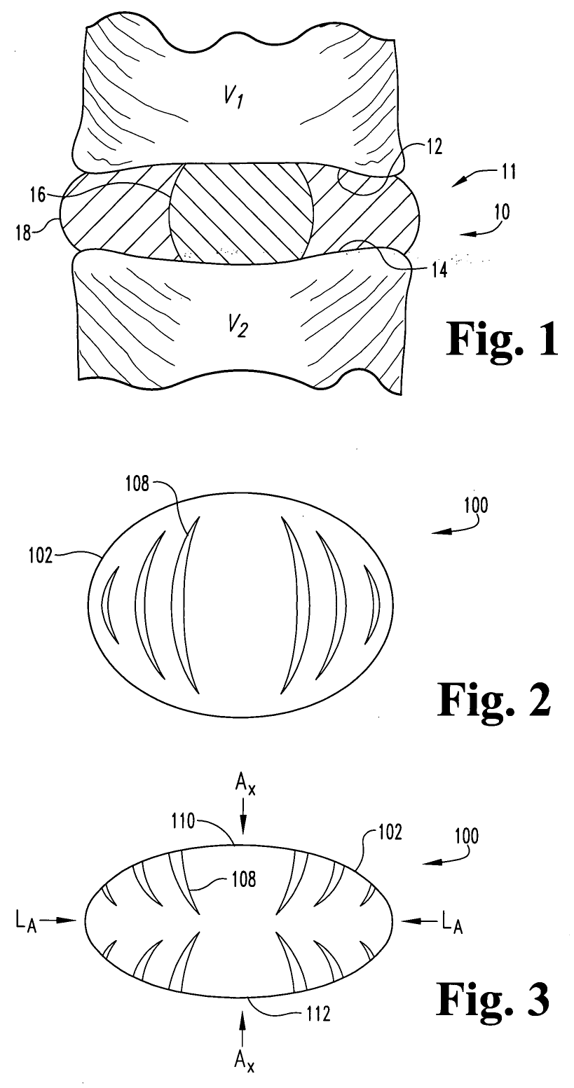

FIG. 1 illustrates a natural or native intervertebral disc 10 positioned in intervertebral disc space 11 between vertebral endplates 12 and 14 of adjacent vertebrae V1 and V2, respectively. Disc 10 includes a nucleus pulposus 16 surrounded by an annulus fibrosis 18. An intervertebral disc, such as the illustrated disc 10, may become displaced or damaged and require removal and replacement of a portion or all of the disc. In certain embodiments, the nucleus pulposus of the intervertebral disc may be removed and replaced with a nucleus replacement implant such as those described herein.

FIGS. 2 and 3 illustrate an embodiment of a nucleus replacement implant 100 to replace a nucleus pulposus of an intervertebral disc. Implant 100 includes a compressive elastic body 102. As illustrated, body 102 can include slots 108 therein to better allow for compression of implant 100. In that embodiment, slots 108 are generally wider in a middle portion and come to points at their ends, and are oriented so that their respective middle portions are generally in superior or inferior parts of body 102 and their respective ends follow the contour of the exterior of body 102 to side portions of body 102. Compression of implant 100 may provide for easier insertion of the implant and increased performance of the implant when implanted in the intervertebral disc space. In other embodiments, slots 108 can be sized, configured and/or arranged differently than as illustrated in FIGS. 2 and 3. Additionally, in certain embodiments, implant 100 could include more or fewer slots 108 than as illustrated. In certain embodiments, it is contemplated that slots 108 are absent from implant 100.

Additionally, implant 100 can include convex superior and inferior surfaces 110 and 112, respectively, to contact vertebral endplates of adjacent vertebrae and provide a better anatomical fit of implant 100 in the intervertebral disc space. In certain embodiments, convex superior and inferior surfaces 110 and 112 may be spherical in shape. Additionally in certain embodiments, convex superior and inferior surfaces 110 and 112 are configured to articulate with vertebral endplates of adjacent vertebrae. It is also contemplated that implant 100 can be compressed both in an axial direction A_{X} and in a lateral direction L_{A}. For purposes of the present disclosure, axial compression includes compression that is generally along or parallel to a longitudinal axis of the spine and lateral compression includes compression that is generally perpendicular to a longitudinal axis of the spine. In such embodiments, elastic body 102 includes a sufficiently low elastic modulus to allow for at least slight compression of implant 100. In the illustrated embodiment, implant 100 is generally saucer shaped; however, it should be appreciated that implant 100 can be configured differently, such as elliptical in shape as an example.

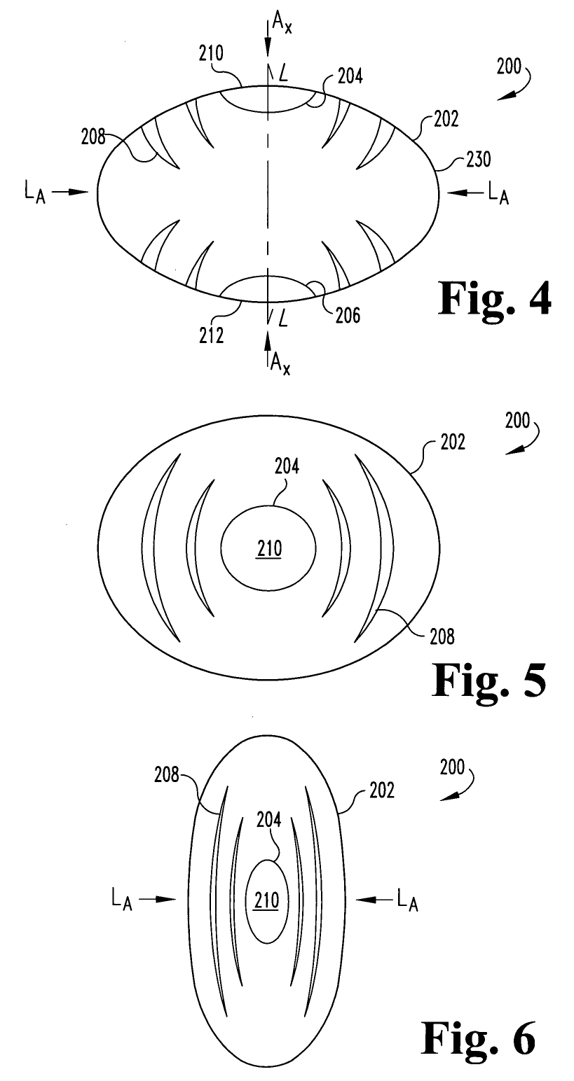

Referring generally to FIGS. 4-6, a nucleus replacement implant 200 similar to implant 100 is illustrated. Implant 200 further includes end portions or end members to contact endplates of adjacent vertebral bodies. Implant 200 includes a compressive elastic body 202 at least partially surrounding superior and inferior end portions 204 and 206, respectively, aligned along a longitudinal axis L. In the illustrated embodiment, elastic body 202 includes slots 208 to assist in compression of implant 200. Compression of implant 200 may allow for easier insertion of implant 200 in the intervertebral disc space and the necessary movement of implant 200 after implantation in the intervertebral disc space in conjunction with movement of the adjacent vertebrae. As stated above in connection with slots 108 in FIGS. 2 and 3, slots 208 could be sized, configured and/or arranged differently, and could number more or less than as in the illustrated embodiment. It is contemplated that in certain embodiments, slots 208 are absent from implant 200.

Superior and inferior end portions 204 and 206 can include convex outer surfaces 210 and 212, respectively. In certain embodiments, surfaces 210 and 212 are spherical and are configured to conform to the shape of the vertebral endplates of the intervertebral disc space in which implant 200 is positioned. In certain embodiments, outer surfaces 210 and 212 are configured to articulate with the vertebral endplates. Additionally, in certain embodiments, end portions 204 and 206 may be substantially thin pieces of material engaged with an outer surface of body 202. In certain other embodiments, end portions 204 and 206 can be substantially surrounded by elastic body 202 and can be shaped in various manners. In some cases, end portions 204 and 206 can be parts of one integral component extending along longitudinal axis L. In other cases, end portions 204 and 206 are separate components with part of elastic body 202 positioned between the end portions to allow for axial compression of implant 200.

In the illustrated embodiment, elastic body 202 includes an exposed outer surface 230. Accordingly, the periphery of implant 200 includes outer surfaces 210 and 212 of end portions 204 and 206 and outer surface 230 of elastic body 202. In certain embodiments, end portions 204 and 206 include a higher elastic modulus than the elastic modulus of body 202, such that elastic body 202 limits the amount of subsidence experienced by implant 200 relative to the adjacent vertebrae in the intervertebral disc space in which implant 200 is positioned. Additionally, it is contemplated that in certain embodiments implant 200 can be compressed both in an axial direction Aₓ and in a lateral direction L_{A}. In such embodiments, elastic body 202 includes a sufficiently low elastic modulus to allow for such compression. In the illustrated embodiment, implant 200 is generally circular or saucer-shaped. However, it should be appreciated that implant 200 can be shaped differently than as illustrated.

FIG. 6 illustrates implant 200 under lateral compression along lateral direction L_{A}, changing the shape of implant 200 to a generally elongate or elliptical shape. In such embodiments, the generally elongate or elliptical shape of implant 200 can assist in insertion and implantation in the intervertebral disc space according to a minimally invasive approach.

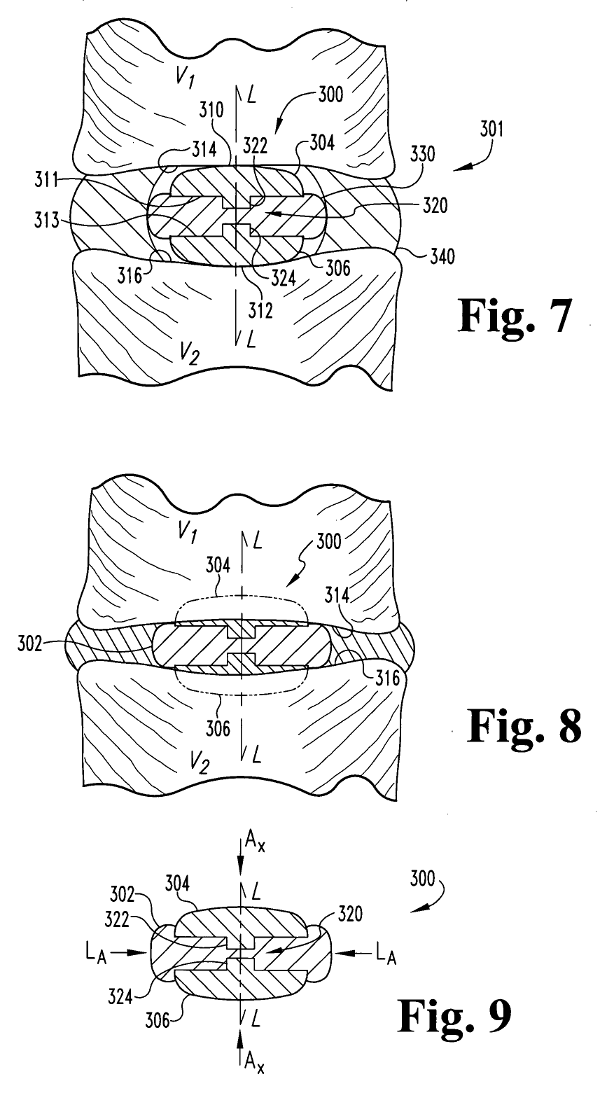

FIGS. 7-9 illustrate a nucleus replacement implant 300 positionable in intervertebral disc space 301 between adjacent vertebrae V1 and V2 to replace a natural nucleus pulposus of an intervertebral disc. Similar to implant 200, implant 300 can include an elastic body 302 at least partially surrounding superior and inferior end potions 304 and 306 aligned along a longitudinal axis L. In certain embodiments, end portions 304 and 306 are configured to contact vertebrae V1 and V2 and assist in compression of implant 300. Superior and inferior end portions 304 and 306 can include convex superior and inferior outer surfaces 310 and 312 and opposing inner surfaces 311 and 313, respectively. In certain embodiments, outer surfaces 310 and 312 may be spherical and configured to conform to the shape of superior and inferior vertebral endplates 314 and 316, respectively, of adjacent vertebrae V1 and V2. Additionally, in certain embodiments, outer surfaces 310 and 312 may be configured to articulate with vertebral endplates 314 and 316.

In the illustrated embodiment, end portions 304 and 306 are separate components with elastic body 302 surrounding part of end portions 304 and 306. Additionally, in the illustrated embodiment, inner surfaces 311 and 313 define a gap 320 and are in contact with elastic body 302 such that part of body 302 is positioned in gap 320, thereby allowing for axial compression of implant 300, as will be discussed in greater detail. In certain embodiments, implant 300 can be compressed both in an axial direction A_{X} and in a lateral direction L_{A}. In such embodiments, elastic body 302 includes a sufficiently low elastic modulus to allow for such compression. In the illustrated embodiment, inner surfaces 311 and 313 define center stumps 322 and 324, respectively. However, it should be appreciated that the inner surfaces can be configured differently. Additionally, in the illustrated embodiment, elastic body 302 includes an exposed outer surface 330 which is annular in shape about longitudinal axis L. Accordingly, the periphery of implant 300 includes outer surfaces 310 and 312 of end portions 304 and 306, respectively, and outer surface 330 of elastic body 302.

As illustrated, implant 300 can be positioned within an annulus fibrosis 340. In certain embodiments, annulus 340 is the natural or native annulus fibrosis from the natural intervertebral disc. In certain other embodiments, annulus 340 is a prosthetic annulus positioned within intervertebral disc space 301. Additionally, it is contemplated that, in certain embodiments, implant 300 is positioned in intervertebral disc space 301 with no annulus fibrosis positioned therein.

As illustrated in FIG. 8, end portions 304 and 306 can include a higher elastic modulus than that of elastic body 302, such that elastic body 302 limits the amount of subsidence experienced by implant 300 relative to adjacent vertebrae V1 and V2. In certain situations, implant 300 may experience subsidence wherein end portions 304 and 306 are compressed into vertebral endplates 314 and 316. In the illustrated embodiment, elastic body 302 extends outward of end portions 304 and 306 transverse to longitudinal axis L, thereby contacting endplates 314 and 316 as illustrated. Elastic body 302 can include a sufficiently low elastic modulus to limit further subsidence experienced by implant 300, such that body 302 is not compressed into endplates 314 and 316.

FIG. 9 illustrates implant 300 under axial compression along axial direction Aₓ. In the illustrated embodiment, end portions 304 and 306 are compressed towards each other, lessening gap 320 between stumps 322 and 324 of end portions 304 and 306. In certain embodiments, the part of body 302 positioned in gap 320 may allow for such axial compression. As illustrated, under axial compression, elastic body 302 may spread further outward of end portions 304 and 306 transverse to longitudinal axis L. It is contemplated that in other embodiments, elastic body 302 can include slots therein to assist in compression of implant 300. Compressibility of implant 300 may allow for easier insertion of the implant in the intervertebral disc space and increased performance of the implant after positioning in the intervertebral disc space.

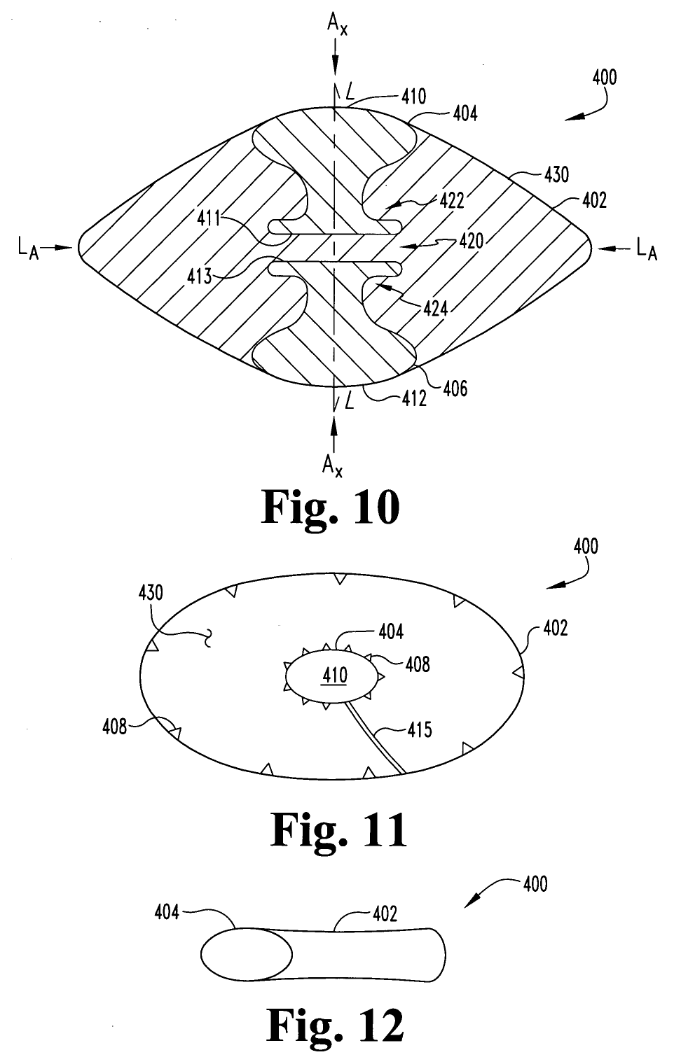

FIGS. 10-12 illustrate a nucleus replacement implant 400 positionable in an intervertebral disc space between adjacent vertebrae to replace the natural nucleus pulposus of an intervertebral disc. Similar to implants 200 and 300, implant 400 includes an elastic body 402 at least partially surrounding superior and inferior end portions 404 and 406 aligned along a longitudinal axis L. In certain embodiments, end portions 404 and 406 may be configured to contact vertebrae and assist in and/or limit the degree of compression of implant 400. End portions 404 and 406 can include superior and inferior convex outer surfaces 410 and 412 and opposing inner surfaces 411 and 413, respectively. In certain embodiments, surfaces 410 and 412 are spherical and are configured to conform to the shape of all or part of vertebral endplates (not shown) of the intervertebral disc space in which implant 400 is positioned. Additionally in certain embodiments, outer surfaces 410 and 412 may be configured to pivot or otherwise articulate with vertebral endplates of adjacent vertebrae.

In the illustrated embodiments, end portions 404 and 406 are separate components, with a substantial part of end portions 404 and 406 surrounded by elastic body 402. Inner surfaces 411 and 413 are in contact with elastic body 402 and define a gap 420 in which part of body 402 is positioned, thereby allowing for axial compression of implant 400, at least to the point where inner surfaces 411 and 413 engage each other or approach closely enough that the portion of body 402 between them is no longer compressible by the applied force. Axial compression of implant 400 can assist in the insertion of implant 400 in an intervertebral disc space. Inner surfaces 411 and 413 in the illustrated embodiment define generally T-shaped configurations 422 and 424, respectively, with T-shaped configuration 422 being inverted in the illustrated embodiment. In certain embodiments, the T-shaped configurations 422 and 424 may assist in maintaining engagement of end portions 404 and 406 with elastic body 402. However, it should be appreciated that end portions 404 and 406 can be in engagement with body 402 in other appropriate manners, including via other appropriate holding or capturing configurations of the end portions.

In the illustrated embodiment, elastic body 402 includes an exposed outer surface 430. Accordingly, the periphery of implant 400 includes outer surfaces 410 and 412 of end portions 404 and 406, respectively, and outer surface 430 of elastic body 402. In certain embodiments, end portions 404 and 406 can be rigid or include a material of higher elastic modulus than elastic body 402 such that elastic body 402 limits the amount of subsidence experienced by implant 400 relative to adjacent vertebrae of the intervertebral disc space in which implant 400 is positioned. As described above in connection with FIG. 8, in certain situations implant 400 can experience subsidence such that end portions 404 and 406 are compressed into intervertebral endplates as a result of axial compression along an axial direction Aₓ. In the illustrated embodiment, elastic body 402 extends outward of end portions 404 and 406 transverse to longitudinal axis L to contact the vertebral endplates and may limit further subsidence of implant 400. Additionally in certain embodiments, implant 400 can be compressed both in axial direction Aₓ and in a lateral direction L_{A}. In such embodiments, elastic body 402 can include a sufficiently low elastic modulus to allow for such compression.

As illustrated in FIG. 11, elastic body 402 can include slots 408 therein to better allow for compression, and folding and unfolding of implant 400. In the illustrated embodiment, slots 408 are configured as relief cuts around body 402 at positions adjacent end portions 404 and 406, and at the position of largest diameter of body 402. In other embodiments, slots 408 could be sized, configured and/or arranged differently than as illustrated in FIG. 11. In certain embodiments, implant 400 can include more or fewer slots 408 than as illustrated. Additionally in certain embodiments, it is contemplated that slots 408 are absent from implant 400.

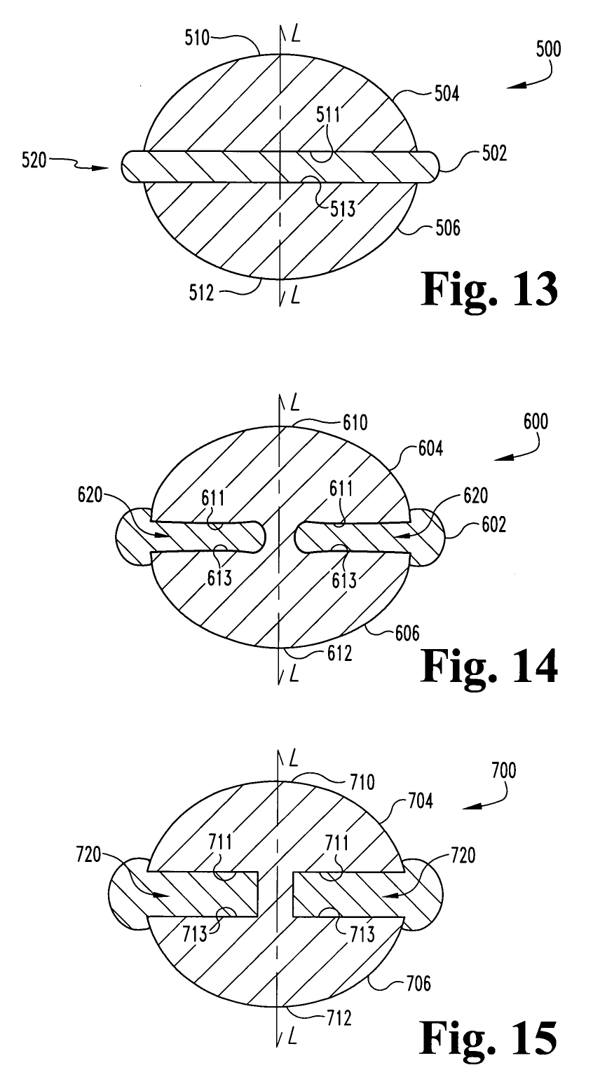

In certain embodiments, implant 400 may include shape memory, allowing for extensive short-term manual or other deformation without permanent deformation, cracks, tears, breakage or other damage. Additionally, body 402 of implant 400 can include a fold line 415 to assist in the folding and unfolding of implant 400. As illustrated in FIG. 12, body 402 is configured in certain embodiments to fold around end portions 404 and 406 to assist in the insertion of implant 400 in an intervertebral disc space, among other things. In certain embodiments, body 402 is composed of a shape-memory polymer which urges body 402 to fold around end portions 404 and 406 as illustrated in FIG. 12. In such cases, body 402 returns by itself, automatically, back into the first, folded or wrapped configuration once manual (e.g. direct compression by the surgeon's hands or tools) or other force is no longer exerted on body 402. In certain other embodiments, body 402 is composed of a shape-memory polymer which urges body 402 to unfold around end portions 404 and 406 to the position illustrated in FIG. 11. Shape memory implant 400 may provide improved handling and manipulation characteristics in that the implant may be deformed, configured and otherwise handled by an individual without resulting in any breakage or other damage to the implant.

Referring generally to FIGS. 13-21, various further embodiments of nucleus replacement implants according to the present disclosure are illustrated. The nucleus replacement implants illustrated in FIGS. 13-21 are configured to be positioned in an intervertebral disc space between adjacent vertebrae to replace a natural nucleus pulposus of an intervertebral disc. The illustrated implants include opposing superior and inferior convex or spherical surfaces configured to contact vertebral endplates of adjacent vertebrae and, in certain embodiments, configured to articulate with the vertebral endplates. The implants illustrated in FIGS. 13-21 generally include end portions (or members) and an elastic body, with the elastic modulus of the body being less that the elastic modulus of the end portions. In certain embodiments, the end portions are part of one integral core component (see FIGS. 14-16 and 18-19), and in certain other embodiments, the end portions are separate individual end members (see FIGS. 13, 17 and 20-21). Although two separate end members may allow for greater axial compression of the nucleus replacement implant, it should be appreciated that in the embodiments having one integral core component with end portions, the core component can be composed of an at least partially flexible material such that at least slight axial compression is possible to assist in the insertion and implantation of the implant in an intervertebral disc space.

Additionally in the illustrated implants, the elastic body of each implant extends outward of the end portions at least one location transverse to a longitudinal axis of the end portions. In this respect, the implants may be configured to at least partially limit the amount of subsidence experienced by the implant. In certain embodiments, the elastic bodies are load-bearing components configured to substantially bear the loads experienced by the particular implant. Additionally in certain embodiments, the elastic bodies of the implants each include a sufficiently low elastic modulus to allow for at least partial axial and/or lateral compression of the particular implant. Compression of the nucleus replacement implants may assist in their insertion and implantation in intervertebral disc spaces. Further, although slots are not illustrated in the embodiments of FIGS. 13-21, it is contemplated that slots can be present in the elastic bodies of one or more of the various embodiments to assist in compression of the corresponding implant(s). The illustrated embodiments are intended to serve as examples of the various possible geometric configurations of nucleus replacement implants according to the present disclosure. It should be appreciated that other appropriate configurations are possible and contemplated.

Referring to FIG. 13, a nucleus replacement implant 500 includes an elastic body 502 positioned between end portions 504 and 506 along a longitudinal axis L. End portions 504 and 506 may include convex outer surfaces 510 and 512, respectively, for contacting a vertebral endplate and inner surfaces 511 and 513, respectively, in contact with elastic body 502. As illustrated, inner surfaces 511 and 513 define a gap 520, with part of elastic body 502 being positioned in gap 520 to allow for compression of implant 500. In the illustrated embodiment, end portions 504 and 506 are generally half circular in shape with elastic body 502 positioned therebetween and extending outward of end portions 504 and 506 transverse to longitudinal axis L to limit subsidence.

FIG. 14 illustrates a nucleus replacement implant 600 according to another embodiment having elastic body 602 at least partially surrounding end portions 604 and 606 positioned along a longitudinal axis L. In the illustrated embodiment, end portions 604 and 606 may include convex outer surfaces 610 and 612, respectively, for contacting a vertebral endplate and inner surfaces 611 and 613 in contact with elastic body 602. Additionally as illustrated, end portions 604 and 606 can be generally hourglass shaped in combination and/or form a generally I-shaped configuration in cross section. In the illustrated embodiment, end portions 604 and 606 define a gap 620 between inner surfaces 611 and 613. Additionally, elastic body 602 may be positioned in gap 620 and extend outward of end portions 604 and 606 transverse to longitudinal axis L to limit subsidence.

FIG. 15 illustrates a nucleus replacement implant 700 having elastic body 702 at least partially surrounding end portions 704 and 706. End portions 704 and 706 can include convex outer surfaces 710 and 712, respectively, for contacting a vertebral endplate and inner surfaces 711 and 713, respectively, in contact with elastic body 702. In the illustrated embodiment, end portions 704 and 706 together form a generally I-shaped configuration in cross section and define a gap 720 between inner surfaces 711 and 713. In the illustrated embodiment, elastic body 702 is positioned in gap 720 and extends outward of end portions 704 and 706 transverse to longitudinal axis L to limit subsidence. Implant 700 is similar in design and function to implant 600, except that inner surfaces 611 and 613 join together in a curved relationship and inner surfaces 711 and 713 include straight segments with substantially 90 degree bend angles.

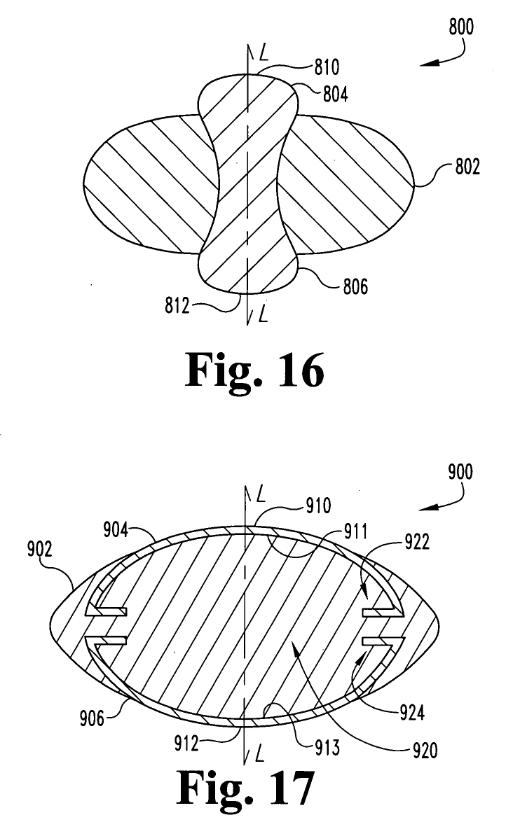

Referring to FIG. 16, there is illustrated a nucleus replacement implant 800 having elastic body 802 at least partially surrounding end portions 804 and 806 positioned along a longitudinal axis L. End portions 804 and 806 may be parts of one integral core member and include convex outer surfaces 810 and 812, respectively, for contacting a vertebral endplate. In the illustrated embodiment, end portions 804 and 806 together form a generally hourglass shape. However, it should be appreciated that end portions 804 and 806 can together form a different configuration. Elastic body 802 may surround part of end portions 804 and 806 and extend outward of end portions 804 and 806 transverse to a longitudinal axis L to limit subsidence.

FIG. 17 illustrates a nucleus replacement implant 900 having elastic body 902 between end portions 904 and 906 positioned along a longitudinal axis L. End portions 904 and 906 may include convex outer surfaces 910 and 912, respectively, for contacting a vertebral endplate and inner surfaces 911 and 913 in contact with elastic body 902. As illustrated, inner surfaces 911 and 913 define a gap 920, with part of elastic body 902 being positioned in gap 20 to allow for compression of implant 900. In the illustrated embodiment, end portions 904 and 906 are generally C-shaped, with elastic body 902 positioned therebetween and extending outward of end portions 904 and 906 transverse to longitudinal axis L to limit subsidence. Additionally, end portions 904 and 906 may optionally include hook segments 922 and 924, respectively, to assist in maintaining engagement of end portions 904 and 906 with elastic body 902. However, it should be appreciated that end portions 904 and 906 can optionally include other configurations to assist in maintaining engagement with elastic body 902.

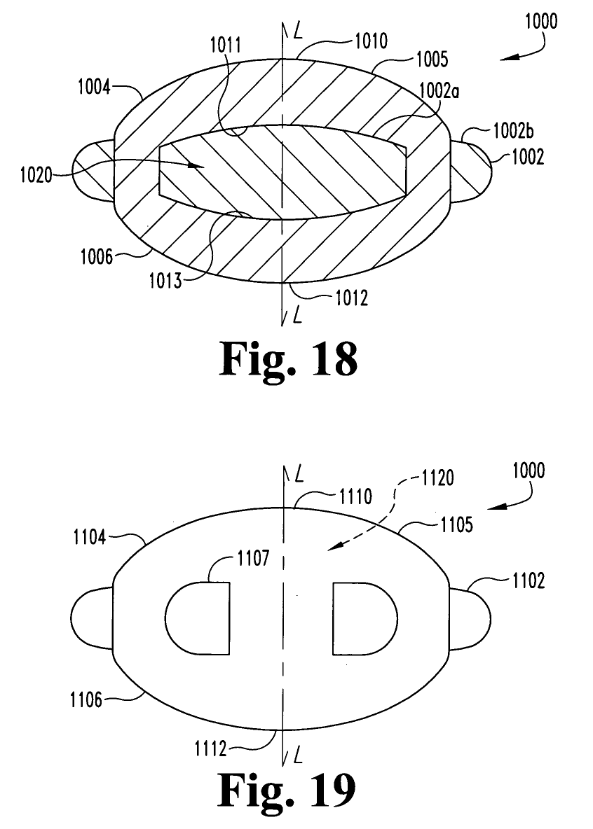

FIG. 18 illustrates a nucleus replacement implant 1000 having elastic body 1002 between end portions 1004 and 1006 positioned along a longitudinal axis L. In the illustrated embodiment, elastic body 1002 includes a center portion 1002a and an outer portion 1002b. In certain embodiments, end portions 1004 and 1006 may be part of a hollow ball or sphere 1005 with elastic body portion 1002a positioned in the center of sphere 1005 and elastic body portion 1002b forming a ring outside of sphere 1005. End portions 1004 and 1006 can include convex outer surfaces 1010 and 1012, respectively, for contacting a vertebral endplate and inner surfaces 1011 and 1013 in contact with elastic body 1002. As illustrated, inner surfaces 1011 and 1013 define a gap 1020 with elastic body portion 1002a being positioned therein. However, it should be appreciated that implant 1000 can be configured differently in accordance with the present disclosure. As an example, implant 1000 can be configured such that elastic body portion 1002a is connected at one or more locations with elastic body portion 1002b.

Referring to FIG. 19, there is shown a nucleus replacement implant 1100, similar to implant 1000, having an elastic body 1102 and a hollow core 1105 with end portions 1104 and 1106 along a longitudinal axis L. In the illustrated embodiment, core 1105 includes openings 1107 in communication with a hollow center 1120, with elastic body 1102 positioned in hollow center 1120 and also extending out openings 1107 transverse to longitudinal axis L to limit subsidence. End portions 1104 and 1006 can include convex outer surfaces 1110 and 1112, respectively, for contacting a vertebral endplate. It should be appreciated that implant 1100 can be configured differently than as illustrated. As an example, openings 1107 can number more or less than the number of openings illustrated in FIG. 19.

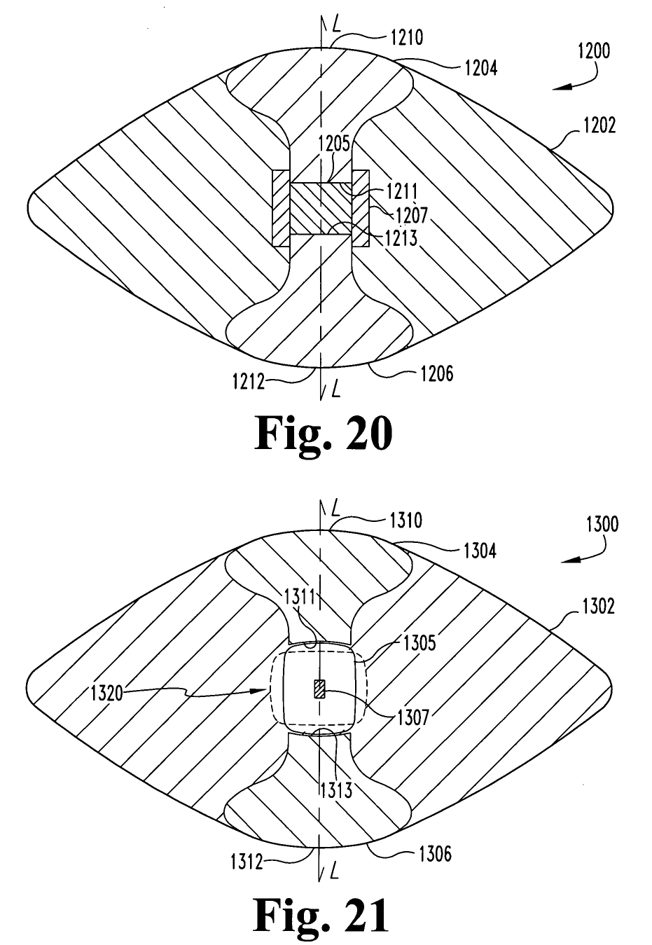

FIG. 20 illustrates a nucleus replacement implant 1200 having an elastic body 1202 at least partially surrounding end portions 1204 and 1206 positioned along a longitudinal axis L. End portions 1204 and 1206 can include convex outer surfaces 1210 and 1212, respectively, for contacting a vertebral endplate and opposing inner surfaces 1211 and 1213, respectively. Implant 1200 may further include an elastic center 1205 at least partially surrounded by a jacket 1207. Elastic center 1205 contacts inner surfaces 1211 and 1213 and allows for axial compression of implant 1200. As described above in connection with FIG. 9, when implant 1200 experiences axial compression, center 1205 will expand outward transverse to longitudinal axis L as inner surfaces 1211 and 1213 are urged towards each other. In such embodiments, jacket 1207 surrounding center 1205 can constrain the amount of compression experienced by center 1205 and limit the amount of axial compression of implant 1200. Accordingly, in certain embodiments, jacket 1207 is composed of a material having a higher elastic modulus than the elastic modulus of center 1205. In the illustrated embodiment, implant 1200 is generally saucer shaped.

A nucleus replacement implant 1300 is illustrated in FIG. 21 and includes an elastic body 1302 at least partially surrounding end portions 1304 and 1306 positioned along a longitudinal axis L. End portions 1304 and 1306 can include convex outer surfaces 1310 and 1312, respectively, for contacting a vertebral endplate and opposing inner surfaces 1311 and 1313, respectively, defining a gap 1320 therebetween. Implant 1300 further includes a rotatable post 1305 defining a tool receiving bore 1307. When post 1305 is positioned in a generally horizontal or lateral position, gap 1320 has at least slight clearance to allow for axial compression of implant 1300. In the illustrated embodiment, post 1305 can be rotated to a generally vertical position such that post 1305 substantially fills gap 1320, thereby substantially preventing axial compression of implant 1300. In the illustrated embodiment, post 1305 is generally rectangular in shape with rounded corners. However, it should be appreciated that post 1305 can be configured differently, such that post 1305 can be rotated to substantially prevent axial compression of implant 1300. Post 1305 can be rotated by inserting the head of an instrument in bore 1307. In certain embodiments, an instrument passageway (not shown) extends from the outer surface of implant 1300 to bore 1307. However, it should be appreciated that other mechanisms of rotating post 1305 can be used. In the illustrated embodiment, implant 1300 is generally saucer shaped; however, it should be appreciated that implant 1300 can be shaped and sized differently.

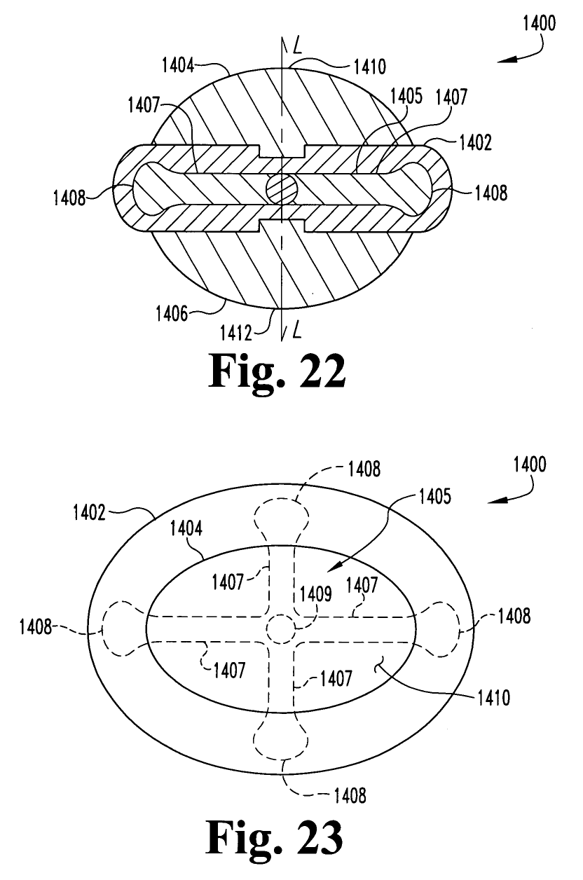

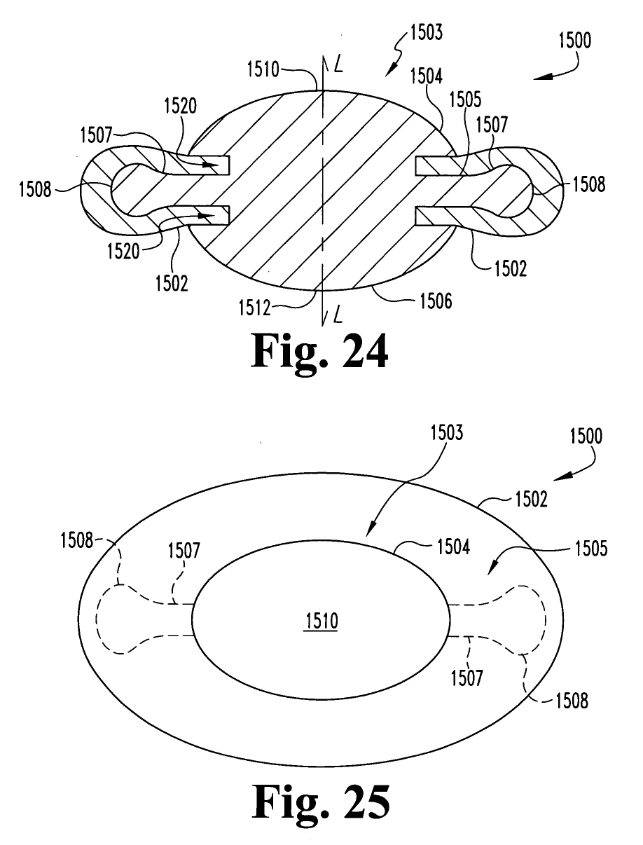

Referring generally to FIGS. 22-25, two additional embodiments of nucleus replacement implants according to the present disclosure are illustrated. The nucleus replacement implants of FIGS. 22-25 are configured to be positioned in an intervertebral disc space between adjacent vertebrae to replace a natural nucleus pulposus of an intervertebral disc. The implants illustrated in FIGS. 22-25 include opposing superior and inferior convex or spherical outer surfaces configured to contact vertebral endplates of adjacent vertebrae and, in certain embodiments, configured to articulate with the vertebral endplates. The illustrated implants generally include end portions (or members), an elastic body and at least one rigid motion limiter, with the elastic modulus of the elastic body being less than the elastic modulus of the end portions and the motion limiter. In the embodiment illustrated in FIGS. 24-25, the end portions are parts of one integral core component, and in the embodiment illustrated in FIGS. 22-23, the end portions are separate individual end members.

Additionally, in the embodiments illustrated in FIGS. 22-25, the elastic body extends outward of the end portions transverse to a longitudinal axis of the end portions. In this respect, the implants may be configured to at least partially limit the amount of subsidence experienced thereby. Additionally in certain embodiments, the elastic bodies of the implants can include a sufficiently low elastic modulus to allow for at least partial axial and/or lateral compression of the particular implant. Compression of the nucleus replacement implants can assist in their insertion and implantation in intervertebral disc spaces. Further, it is contemplated that slots can be present in the elastic bodies of the implants to assist in the compression thereof. The illustrated embodiments are intended to serve as examples of the various possible configurations of nucleus replacement implants having rigid motion limiters according to the present disclosure. It should be appreciated that other appropriate configurations including rigid motion limiters are possible and contemplated.

Referring more specifically to FIGS. 22-23, nucleus replacement implant 1400 includes elastic body 1402 positioned between end portions 1404 and 1406 along a longitudinal axis L. End portions 1404 and 1406 can include convex superior and inferior outer surfaces 1410 and 1412, respectively, configured to contact adjacent vertebral endplates in an intervertebral disc space. Implant 1400 may further include a rigid motion limiter 1405. Implant 1400 is similar in structure and function to implant 300 illustrated in FIGS. 7-9, with implant 1400 including a rigid motion limiter 1405. Accordingly, much of the description of implant 300 applies to implant 1400 as well and will not be repeated herein for the sake of brevity. As can be seen from a top view of implant 1400 in FIG. 23, motion limiter 1405 can include four equally spaced apart arms 1407 extending outward from longitudinal axis L. Additionally, arms 1407 can optionally include rounded ends 1408 and define a center hole 1409. Center hole 1409 can allow for axial compression of implant 1400 in that end portions 1404 and 1405 can compress towards each other via hole 1409. It is contemplated that motion limiter 1405 can be configured and sized differently. As an example, rather than four separate arms, motion limiter 1405 could extend continuously about longitudinal axis L, or could more or fewer than four separate arms. In certain embodiments, motion limiter 1405 includes a higher elastic modulus than elastic body 1402. Additionally in certain embodiments, motion limiter 1405 can include a sufficiently high elastic modulus such that motion limiter 1405 prevents excessive and/or undesired compression, bending or rotation of implant 1400.

Referring to FIGS. 24-25, there is shown a nucleus replacement implant 1500 having elastic body 1502 and core member 1503 having end portions 1504 and 1506 positioned along a longitudinal axis L. End portions 1504 and 1506 can include convex superior and inferior outer surfaces 1510 and 1512, respectively, configured to contact adjacent vertebral endplates in an intervertebral disc space. Implant 1500 may further include a motion limiter 1505 between end portions 1504 and 1506. In the illustrated embodiment, motion limiter 1505 is not a separate component, as in implant 1400, but rather is integral with end portions 1504 and 1506 as part of core member 1503. Additionally, in the illustrated embodiment, core member 1503 defines gaps 1520 between each of end portions 1504 and 1506 and motion limiter 1505, with elastic body 1502 being positioned in gaps 1520 and surrounding motion limiter 1505. As can be seen from a top view of implant 1500 in FIG. 25, motion limiter 1505 can include four equally spaced apart arms 1507 extending outward from longitudinal axis L. Additionally, arms 1507 can optionally include rounded ends 1508. It is contemplated that motion limiter 1505 can be configured differently. As an example, motion limiter 1505 could extend continuously about longitudinal axis L, or can include more or fewer than four arms. In certain embodiments, core member 1503 includes a higher elastic modulus than elastic body 1502. Additionally in certain embodiments, motion limiter 1505 (and the remainder of core 1503) includes a sufficiently high elastic modulus such that motion limiter 1505 prevents excessive or undesired compression, bending and/or rotation of implant 1500.

Referring generally to implants 100, 200, 300, 400, 500, 600, 700, 800, 900, 1000, 1100, 1200, 1300, 1400 and 1500, the elastic bodies therein can be composed of a wide variety of biocompatible polymeric materials, including elastic materials, such as elastomeric materials, hydrogels or other hydrophilic polymers, or composites thereof. For example, the elastic bodies can be composed of an elastomer such as silicone, polyurethane, copolymers of silicone and polyurethane, polyolefins, nitrile and any combinations thereof. Examples of polyurethanes include thermoplastic polyurethanes, aliphatic polyurethanes, segmented polyurethanes, hydrophilic polyurethanes, polyether-urethane, polycarbonate-urethane and silicone polyether-urethane. In certain embodiments, the elastic bodies can be composed of pursil, a combination of polyurethane and silicone. The nature of the materials employed to form the elastic bodies can be selected so the formed implants have sufficient load bearing capacity.

The end portions of the implants described herein can be composed of a rigid or flexible metal material in certain embodiments. In certain other embodiments, the end portions described herein can be composed of a plastic material. It is contemplated that the end portions can be composed of other appropriate materials such that the end portions include a higher elastic modulus and are therefore less elastic than the corresponding elastic body of the corresponding implant. Additionally, it should be appreciated that the illustrations herein are only few examples of the numerous different geometric possibilities of nucleus replacement implants according to the present disclosure. Further, features of certain implants can be used and incorporated into other implants in combinations not shown.

Referring generally to FIGS. 2-25, the implantation, operation and use of nucleus replacement implants 100, 200, 300, 400, 500, 600, 700, 800, 900, 1000, 1100, 1200, 1300, 1400 and 1500 discussed and illustrated herein will be described with reference to a surgical procedure involving a section of spine. It should be appreciated that the methods described herein involve the use of one or more of the nucleus replacement implants discussed and illustrated herein. It will also be appreciated that other uses of the implants described herein and other surgical procedures can be made.

To treat the condition or injury of the patient, the surgeon obtains access to the surgical site in any appropriate manner, e.g. through incision and retraction of tissues. It is contemplated that the nucleus replacement implants discussed herein can be used in minimally-invasive surgical techniques where the disc space is accessed through a micro-incision, a sleeve, or one or more retractors that provide a protected passageway to the disc space. The implants discussed herein also have application in open surgical techniques where skin and tissue are incised and retracted to expose the surgical site.

Once access to the surgical site has been obtained, e.g. via an opening such as a midline incision above the affected area, with tissue being resected, or by other surgical procedure, and prior to positioning the nucleus replacement implant in the intervertebral disc space, an incision may be made in the annulus fibrosis, or access may made through a defect, deterioration, or other injury in the annulus fibrosis, in order to remove the natural nucleus pulposus and any free disc fragments within the intervertebral disc space. Additionally, the intervertebral disc space may be distracted to a desired level. Once formed, and after preparing the disc space for receiving the nucleus replacement implant, the surgeon may implant the nucleus replacement implant into the intervertebral disc space utilizing one or more appropriate implantation devices. The elastic and compressive nature of the nucleus replacement implants described herein assists in their implantation in the intervertebral disc space. In certain embodiments, the surgeon may manually or by other force compress the particular implant such that the implant can more easily be inserted into the intervertebral disc space via a minimal access surgical approach. As noted previously, the more rigid or flexible end parts, if present, abut the endplates of vertebrae and/or are placed or fitted in hollows or grooves made in endplates or other tissue. Additionally, the elastic and compressive nature of the implants described herein may allow the implants to move in conjunction with movement of the corresponding spinal segment to substantially mimic the function of the native nucleus, thus increasing their performance after implantation in the intervertebral disc space.

While the disclosure has been illustrated and described in detail in the drawings and foregoing description, the same is to be considered as illustrative and not restrictive in character, it being understood that only certain embodiments have been shown and described and that all changes and modifications that come within the spirit of the disclosure are desired to be protected.

What is claimed is:

1. An intervertebral disc nucleus replacement implant for positioning between adjacent vertebrae of a spinal segment, comprising:
opposing superior and inferior end portions substantially aligned along a longitudinal axis, each having an at least partially convex implant-periphery surface for contacting respective endplates of the adjacent vertebrae; and
at least one elastic body surrounding part of each of said end portions and including at least one implant-periphery surface, said body being at least partially compressible, wherein the implant includes an outer periphery comprising said implant-periphery surfaces of said end portions and said implant-periphery surface of said body;
wherein the elastic modulus of said body is lower than the elastic modulus of each of said end portions, and wherein said body extends outward of at least part of each of said end portions in a direction transverse to said longitudinal axis, such that said body is configured to limit the amount of subsidence of the implant relative to the adjacent vertebrae.

2. The implant of claim 1, wherein said end portions are each composed of a metal material.

3. The implant of claim 1, wherein said end portions are each composed of a plastic material.

4. The implant of claim 1, wherein said end portions are separate components.

5. The implant of claim 4, wherein each of said end portions includes a holding configuration to maintain engagement of each of said end portions to said body.

6. The implant of claim 1, comprising a core component, wherein said end portions are portions of said core component.

7. The implant of claim 1, wherein said implant-periphery surface of said body includes an annular shape about said longitudinal axis.

8. The implant of claim 1, wherein each of said end portions includes an inner surface, said inner surface of said superior end portion substantially facing said inner surface of said inferior end portion, wherein said elastic body includes a portion disposed between said inner surfaces to allow for axial compression of the implant.

9. The implant of claim 1, wherein said body is composed of a hydrogel material.

10. The implant of claim 1, wherein said body is composed of an elastomer.

11. The implant of claim 10, wherein said elastomer is selected from the group consisting of silicone, polyurethane, copolymers of silicone and polyurethane, polyolefins, nitrile and combinations thereof.

12. The implant of claim 1, wherein said body includes at least one slot to assist in compression of the implant.

13. The implant of claim 1, wherein each of said implant-periphery surfaces of said end portions is configured to articulate with the respective endplate of the adjacent vertebrae.

14. The implant of claim 1, comprising at least one rigid motion limiter disposed within said body and positioned substantially between said end portions to limit motion of the implant.

15. The implant of claim 1, wherein the implant is configurable in a first wrapped position with said elastic body at least partially wrapped around said end portions and a second expanded position with said elastic body substantially unwrapped around said end portions, wherein said elastic body is composed of a shape memory polymer such that said elastic body recoils to said first wrapped position from said second expanded position.

16. The implant of claim 1, comprising an elastic center portion disposed between said end portions and at least partially surrounded by a constraining jacket configured to constrain the amount of axial compression of said elastic center portion, wherein said elastic center portion and said jacket are disposed within said body.

17. The implant of claim 1, comprising a central locking portion disposed between said end portions, wherein said central locking portion is substantially rectangular in shape and includes a longitudinal axis, said central locking portion being positionable in a first position with said longitudinal axis substantially perpendicular to said longitudinal axis of said end portions and a second position with said longitudinal axis substantially aligned with said longitudinal axis of said end portions, wherein said central locking portion is configured to be rotated from said first position allowing axial compression of the implant, to said second position substantially preventing axial compression of the implant.

18. An intervertebral disc nucleus replacement implant for positioning between adjacent vertebrae of a spinal segment, comprising:
a superior member and an inferior member substantially aligned along a longitudinal axis, and a compressible, elastic body positioned therebetween to allow for axial compression of the implant, each of said superior and inferior members having an inner surface in contact with said body and an opposing at least partially convex outer surface for contacting a respective endplate of the adjacent vertebrae, said elastic body including an annular outer surface, wherein the implant includes an outer periphery comprising said outer surfaces of said superior and inferior members and said outer surface of said body; and
wherein the elastic modulus of said body is lower than the elastic modulus of each of said superior and inferior members, and wherein said body extends outward of at least part of each said superior and inferior members in a direction transverse to said longitudinal axis, such that said body is configured to limit the amount of subsidence of the implant relative to the adjacent vertebrae.

19. The implant of claim 18, wherein said superior and inferior members are each composed of a metal material.

20. The implant of claim 18, wherein each of said superior and inferior members includes an inner capture configuration configured to engage each of said members to said body.

21. The implant of claim 18, wherein said body is composed of an elastomer.

22. The implant of claim 18, wherein said body includes at least one slot to assist in compression of the implant.

23. The implant of claim 18, wherein each of said outer surfaces of said superior and inferior members is configured to articulate with the respective endplate of the adjacent vertebrae.

24. A method for implanting an intervertebral disc nucleus implant in an intervertebral disc space, comprising:
providing an elastic load-bearing nucleus replacement implant, wherein said implant includes an elastic body at least partially surrounding opposed superior and inferior members each having a spherical articulation surface to contact a vertebral endplate, wherein said superior and inferior members are aligned along a longitudinal axis and each include an inner surface opposite said respective articulation surface, with at least part of said elastic body positioned between said inner surfaces to allow for compression of said implant, wherein the elastic modulus of said elastic body is lower than the elastic modulus of each of said superior and inferior members;
compressing said implant to assist in insertion of said implant in the intervertebral disc space, wherein said compressing includes urging at least one of said superior and inferior members toward the other of said superior and inferior members; and
positioning said implant in the intervertebral disc space, including positioning said articulation surfaces in contact with the vertebral endplates.

25. The method of claim 24, wherein said elastic body includes at least one slot to assist in said compressing.

26. The method of claim 24, comprising preparing the intervertebral disc space to receive said implant.

27. The method of claim 24, wherein said elastic body extends outward of said superior and inferior members in a direction transverse to said longitudinal axis, such that said elastic body is configured to limit the amount of subsidence of said implant in the vertebral endplates.
